(19) United States
(12) Patent Application Publication
(10) Pub. No.: US 2023/0349891 A1
(43) Pub. Date: Nov. 2, 2023

(54) RELEASABLE REAGENTS FOR CELL SEPARATION

(71) Applicant: Miltenyi Biotec B.V. & Co. KG, Bergisch Gladbach (DE)

(72) Inventors: Christian DOSE, Bergisch Gladbach (DE); Mario Assenmacher, Bergisch Gladbach (DE); Philipp Steinbruck, Bergisch Gladbach (DE); Jonathan FAUERBACH, Bergisch Gladbach (DE)

(73) Assignee: Miltenyi Biotec B.V. & Co. KG, Bergisch Gladbach (DE)

(21) Appl. No.: 18/020,650

(22) PCT Filed: Feb. 15, 2022
(86) PCT No.: PCT/EP2021/071980

§ 371 Date: Feb. 10, 2023

(30) Foreign Application Priority Data
Aug. 27, 2020 (EP) EP20193006

(51) Int. Cl. G01N 33/543; G01N 33/535

(52) U.S. Cl. CPC G01N 33/535; G01N 33/54306

(57) ABSTRACT

The invention is directed to a conjugate complex for detecting a target moiety in a sample of biological specimens having the general formula (I) An - Bm^{...}Cq-Xo (I) with A: antigen recognizing moiety; B: first binding moiety C second binding moiety X: detection moiety; n, m, q, o integers between 1 and 100, wherein B and C are non-covalently bound to each other characterised in that B comprises a thiamine unit and C is a moiety recognizing thiamine. Futher, the invention is directed to a method detecting a target moiety in a sample of biological specimens with a conjugate complex having the general formula (I).

### BACKGROUND

The present invention is directed to a process for detection and/or separation of one or multiple target moieties on one or multiple cell populations of interest, in a sample of biological specimens by labelling the target moiety on cells of interest with a conjugate comprising a disruptible unit between an antigen recognizing moiety and a detection moiety.

Cell detection and separation techniques, e.g., magnetic cell separation, flow cytometry or flow sorting, are fundamental tools that contributed to the progress of biomedical research and cellular therapy in the past years. The techniques combine the specific labelling of a target moiety with conjugates having a detectable unit like a magnetic particle to retain and therefore isolate cells in a magnetic field, or like a fluorescent dye or transition metal isotope mass tag to detect and characterize cells by microscopy or cytometry. A technological challenge is still the release of the labelling after detection of the target moiety. Downstream applications like sequential sorting strategies, molecular diagnostic, or cell analysis can be prevented or affected by residual labelling.

Reversible labelling systems are known and for example disclosed in US20080255004, EP2725359, WO 96/31776, US7776562, US8298782 or US9023604. These publications are directed to degradation of the label after detection by enzymes or competitive reaction. Depending on the antigen recognizing moiety, both variants may result in a complete removal of the label for the target cell.

In EP3037821, the variants are combined by using a conjugate for labelling comprising an enzymatic degradable spacer and a tag/anti-tag unit like Biotin/anti-Biotin which can be cleaved by a competitive reaction for example with Streptavidin.

So called releasable conjugates comprising the system Biotin/anti-Biotin are known, but have the disadvantage that Biotin is a compound found in many organisms which may result in undesired release and re-binding (so called “cross talk”). Accordingly, it would be beneficial to use a different tag/anti-tag system which make no use of biotin and/or can be used as an orthogonal tag/anti-tag system in conjunction with the known the system Biotin/anti-Biotin.

Surprisingly it was found that thiamine (vitamin B1) and anti-thiamine can be utilized as such tag/anti-tag systems as thiamine/anti-thiamine has similar binding characteristic as Biotin/anti-Biotin but without crosstalk or cross-reactions.

### SUMMARY

It was therefore an object of the invention to provide conjugates and methods for specific labelling, detection and de-labelling of multiple target moieties simultaneously in a sample of biological specimen in order to enable further labelling and detection strategies.

Object of the invention is therefore a conjugate complex for detecting a target moiety in a sample of biological specimens having the general formula (I)

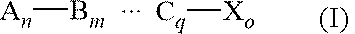

with A: antigen recognizing moiety;
B: first binding moiety
C second binding moiety
X: detection moiety;
n, m, q, o integers between 1 and 100,
wherein B and C are non-covalently bound to each other characterised in that B comprises a thiamine unit and C is a moiety recognizing thiamine.

The general concept of the invention is shown in FIG. 1. This scheme describing the binding, detection and release process of target cells using a conjugate comprised of an antigen recognition moiety and a detection moiety as described in formula I. In short, target cells present in a biological sample are incubated with the detection conjugate. After a period of incubation ranging from 1 to 30 min, the target cells become labeled and can then be detected and/or isolated. After that, upon incubation with free binding reagent, the non-covalent bond between B and C moieties is dissociated and cell lose the detection moiety.

In the following the terms “conjugate complex” and “conjugate” are exchangeable and refer to compounds as defined in the claims comprising covalent and non-covalent bounds.

Further object of the invention is a method for detecting a target moiety in a sample of biological specimens by:
a) providing at least one conjugate complex according to the invention to the sample
b) labelling the target moiety recognized by the antigen recognizing moiety A with at least one conjugate
c) detecting the labelled target moiety via detecting moiety X
d) disrupting the non-covalent bond between Bₘ and C_{q} by adding a release agent that binds to the first binding moiety B, thereby displacing the second binding moiety C and/or by adding a release agent that binds to the second binding moiety C, thereby displacing the first binding moiety B.

The method of the invention may be utilized not only for detecting target moieties i.e. target cells expressing such target moieties, but also for isolating the target cells from a sample of biological specimens. The isolating procedures makes use of detecting the target moieties. For example, the detection of a target moiety by fluorescence may be used to trigger an appropriate separation process as performed on FACS or TYTO (Miltenyi Biotec B.V. & Co. KG) separation systems. In the method of the invention, the well-known magnetic cell separation process can also be used as detection and separation process, wherein the magnetic particles are detected and separated by a magnetic field.

Anti-Thiamine antibodies are commercially available, for example as rat polyclonal antibody from Abcom plc. However, preferable, human monoclonal Anti-Thiamine antibodies are used.

### BRIEF DESCRIPTION OF THE DRAWINGS

FIG. 1 shows the general concept of the method of the invention

FIG. 2 shows affinity systems optional used in the method of the invention

FIGS. 3 and 4 show dot-blots of the cell targeting experiments according to Example 1

### DETAILED DESCRIPTION

The term “disrupting the non-covalent bond between Bm and Cq” means that the non-covalent bond between B and C is abrogated and the binding moiety Cq and detection moiety Xo are removed as fragment Cq-Xo for example by washing.

The method of the invention may involve the removal of the recognizing moiety Aₙ not only from the conjugate, but also from the target moiety. In this respect, the invention encompasses two embodiments by using conjugates with high-affinity (a) or low-affinity (b) antigen recognizing moieties A. A high-affinity antigen recognizing moiety A is capable of binding a target moiety in a 1:1 ratio, i.e. n=1 in formula (I). On the other hand, low-affinity antigen recognizing moieties are not capable of binding a target moiety in a 1:1 ratio, but several low-affinity antigen recognizing moieties in one conjugate are needed to bind to the target moiety, i.e. n>1, preferable n = 2-5 in formula (I).

In a variant of the invention, low-affinity antigen recognizing moieties are multimerized on a dextran backbone. In this variant, a detrain linker is provided between A and B.

The process of the invention may be performed in one or more sequences of the steps a) to d). After each sequence, the detection moiety and optionally the antigen recognizing moiety is released (removed) from the target moiety. Especially when the biological specimens are living cells which shall be further processed, the method of the invention has the advantage of providing unlabelled cells.

After and/or before each step a) - d), one or more washing steps can be performed to remove unwanted material like unbound conjugate (I) or released parts of the conjugate like the binding moiety C and detection moiety X or antigen recognizing moiety A or reagents used for disruption. The term “washing” means that the sample of biological specimen is separated from the environmental buffer by a suitable procedure, e.g., sedimentation, centrifugation, draining or filtration. Before this separation washing buffer can be added and optionally incubated for a period. After this separation, the sample can be filled or resuspended again with buffer.

### Target Moiety

The target moiety to be detected with the method of the invention can be on any biological specimen, like tissues slices, cell aggregates, suspension cells, or adherent cells. The cells may be living or dead. Preferable, target moieties are antigens expressed intracellular or extracellular on biological specimen like whole animals, organs, tissues slices, cell aggregates, or single cells of invertebrates, (e.g., Caenorhabditis elegans, Drosophila melanogaster), vertebrates (e.g., Danio rerio, Xenopus laevis) and mammalians (e.g., Mus musculus, Homo sapiens).

### Detection Moiety

The detection moiety X of the conjugate may be any moiety possessing a property or function which can be used for detection purposes of cells. Preferable, detection moiety X is selected from the group consisting of chromophore moiety, fluorescent moiety, phosphorescent moiety, luminescent moiety, light absorbing moiety, radioactive moiety, transition metal and isotope mass tag moiety, solid support with shape of particles, for example, sheets, plates, membranes, tubes, columns, wells, or micro arrays, magnetic particle.

Suitable fluorescent moieties are those known from the art of immunofluorescence technologies, e.g., flow cytometry or fluorescence microscopy. In these embodiments of the invention, the target moiety labelled with the conjugate is detected by exciting the detection moiety X and detecting the resulting emission (photoluminescence). In this embodiment, the detection moiety X is preferable a fluorescent moiety.

Useful fluorescent moieties might be protein-based, such as phycobiliproteins, polymeric, such as polyfluorenes, small organic molecule dyes, such as xanthenes, like fluorescein, or rhodamines, cyanines, oxazines, coumarins, acridines, oxadiazoles, pyrenes, pyrromethenes, or metallo-organic complexes, such as Ru, Eu, Pt complexes. Besides single molecule entities, clusters of fluorescent proteins or small organic molecule dyes, as well as nanoparticles, such as quantum dots, upconverting nanoparticles, gold nanoparticles, dyed polymer nanoparticles can also be used as fluorescent moieties.

Another group of photo luminescent detection moieties are phosphorescent moieties with time-delayed emission of light after excitation. Phosphorescent moieties include metallo-organic complexes, such as Pd, Pt, Tb, Eu complexes, or nanoparticles with incorporated phosphorescent pigments such as lanthanide doped SrAl₂O₄.

In another embodiment of the invention the target labeled with the conjugate is detected without prior excitation by irradiation. In this embodiment, the detection moiety can be a radioactive label. They may be in the form of radioisotope labelling by exchanging non-radioactive isotopes for their radioactive counterparts, such as tritium, ³²P, ³⁵S or ¹⁴C, or introducing covalently bound labels, such as ¹²⁵I, which is bound to tyrosine, ¹⁸F within fluorodeoxyglucose, or metallo-organic complexes, i.e. ⁹⁹Tc-DTPA.

In another embodiment the detection moiety is capable of causing chemo luminescence, i.e. horseradish peroxidase label in the presence of luminol.

In another embodiment of the invention the target labeled with the conjugate is not detected by radiation emission, but by absorption of UV, visible light, or NIR radiation. Suitable light-absorbing detection moieties are light absorbing dyes without fluorescence emission, such as small organic molecule quencher dyes like N-aryl rhodamines, azo dyes, and stilbenes.

In another embodiment, the light-absorbing detection moieties X can be irradiated by pulsed laser light, generating a photoacoustic signal.

In another embodiment of the invention the target labeled with the conjugate is detected by mass spectrometric detection of a transition metal isotope. Transition metal isotope mass tag labels might be introduced as covalently bound metallo-organic complexes or nanoparticle component. Known in the art are isotope tags of lanthanides and adjacent late transition elements.

Furthermore, the detection moiety X can be a solid support possessing a property or function which can be used for detection purposes of cells. Suitable solid supports are known in biotechnology for immobilizing cells and can have the shape of particles, for example, sheets, plates, membranes, tubes, columns, wells, or micro arrays manufactured from various materials like polystyrene (PS), polymethylmethacrylate (PMMA), polyvinyl toluene (PVT), polyethylene (PE), or polypropylene (PP). Suitable materials are commercially available.

The solid support can further be a magnetic particle, also known in the art as nano- to microscale magnetic bead. The mean diameter of the beads can range from 10 nm to 10 µm. Biocompatible magnetic particles are commercially available and consist of, for example, forms of magnetically iron oxide coated by a shell of dextran molecules or silica. The solid support may also be polymers containing magnetic materials. Suitable particles are commercially available from Miltenyi Biotec B.V. & Co. KG, Germany under the trade name “MicroBeads” and “MACSiBeads” possessing a hydrodynamic diameter of 50-100 nm or 3-4 µm, respectively.

The detection moiety X can be covalently or non-covalently coupled to the binding moiety C. Methods for covalently or non-covalently conjugation is known by persons skilled in the art. In case of a covalent bond between the detection moiety X and the binding moiety C, a direct reaction of an activated group either on the detection moiety X or on the binding moiety C with a functional group on either the binding moiety C or on the detection moiety X or via a heterobifunctional linker molecule, which is firstly reacted with one and secondly reacted with the other coupling partner is possible.

The conjugate used in the method of the invention may comprise 1 to 100, preferable 1 - 20 detection moieties X (i.e. o = 1 to 100, preferable o = 1 - 20).

### Binding Moieties B and C

Binding moiety B and binding moiety C are binding partners capable to bind non-covalently and reversibly to each other. For the purpose of the present invention, non-covalent and reversible bonds are defined as bonds with a dissociation constant of greater than 10E-9 M.

In the conjugate of the invention, binding moiety B comprises a thiamine unit or a unit having a thiamine derivate. Thiamine is also known as “vitamin B12” and has the IUPAC name “2.[3-[(4-amino-2-methylpyrimidin-5-yl)methyl]-4-methyl-1,3-thiazol-3-ium-5-yl]ethanol”.

Accordingly, binding moiety C is a thiamine recognizing moiety binding to B with a dissociation constant greater than 10E-9 M.

In the conjugate of the invention, binding moiety B may stand for either a unit comprising thiamine or a thiamine recognizing moiety as long as binding moiety C is standing for the appropriate binding partner. In other words, if binding moiety B stands for a unit comprising thiamine, C is a thiamine recognizing moiety or if binding moiety B stands for a thiamine recognizing moiety, C is a unit comprising thiamine.

Thiamine itself is known with the following structure and may in a first variant be bound to the antigen recognizing moiety A via its terminal alcohol group.

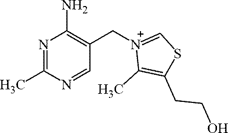 [SMILES: Cc1ncc(C[n+]2csc(CCO)c2C)c(N)n1]

In another variant, the first binding moiety B comprises a thiamine unit according to formula IIa

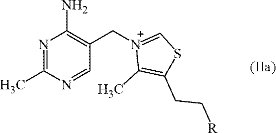

with R representing a spacer group selected from the group consisting of LC, LCLC, PEG, peptide, amino acid, protein, antibody, antibody fragment, fluorescent protein, nanobody and interleukine. The Thiamine unit according to formula II may be bound to the antigen recognizing moiety A via spacer group R. The length of the spacer group R is not particular important, for example PEG can be utilized with 2 to 25 glycol repeating units. The conjugatemay be provided with any kind of counterion like tetrafluoroborate or chlorine. In a concrete example, the first binding moiety B comprises a thiamine according to formula IIb

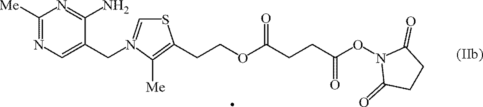

### Disruption of the Non-Covalent Bond Between the Binding Moieties B and C

The non-covalent bond between B and C is reversible and therefore can be disrupted by the addition of competing molecules as release reagent capable of binding to one of the binding moieties B and/or C displacing the respective moiety. Regarding the exemplary mentioned binding moieties in “B and C as binding moieties” competing molecules may be bound or unbound thiamine or analogues and derivatives; pyridoxin or analogues and derivatives; ascorbic acid; folic acid; vitamin B12; vitamin E or analogues and derivatives; a unit comprising thiamine peptides like polyhistidine, FLAG-peptide, calmodulin-binding peptide; small molecules like imidazole or maltose; chelators like EDTA; polymers like polyethylene glycol; complementary oligonucleotides.

The efficiency of the competing reaction is dependent on the thermodynamic and kinetic characteristic of the interaction between the binding moieties B and C and the competing molecule, the concentration of the components, the environmental conditions like temperature, pH and the reaction time. The specific conditions have to be evaluated according to the desired efficiency.

In a variant of the invention the competing molecule displacing the binding moiety C may be conjugated to a different detection moiety X therefore enabling during disruption an exchange of the detection moiety.

Furthermore, the disruption may be achieved by the initiation of conformational changes lowering the strength of the binding interaction between the binding moieties B and C, e.g., chelating divalent cation bound in calmodulin inducing conformational change.

Beside this, the bond between B and C may be cleaved by mechanical agitation inducing shear forces or by changing environmental conditions like pH, temperature or salt concentrations influencing the binding interaction.

It is possible to combine more than one method for disruption.

Usually the efficiency of the cleavage induces a reduction of the labelling with the C-X of at least about 80%, more usually of at least about 95%, preferably of at least about 99%. The conditions for release may be empirically optimized in terms of temperature, pH, etc. The disruption will usually be completed in at least about 15 minutes, more usually at least about 10 minutes, and will usually not be longer than about 2 h.

### Antigen Recognizing Moiety A

The term “antigen recognizing moiety A” refers to any kind of antibody, fragmented antibody or fragmented antibody derivatives, directed against the target moieties expressed on the biological specimens, like antigens expressed intracellular or extracellular on cells. The term relates to an antibody, a fragmented antibody, a fragmented antibody derivative, peptide/MHC-complexes targeting TCR molecules, cell adhesion receptor molecules, receptors for costimulatory molecules or artificial engineered binding molecules. Fragmented antibody derivatives, are for example Fab, Fab′, F(ab′)2, sdAb, scFv, di-scFv, nanobodies. Such fragmented antibody derivatives may be synthesized by recombinant procedures including covalent and non-covalent conjugates containing these kind of molecules. Further examples of antigen recognizing moieties are peptide/MHC-complexes targeting TCR molecules, cell adhesion receptor molecules, receptors for costimulatory molecules, artificial engineered binding molecules, e.g., peptides or aptamers which target, e.g., cell surface molecules.

The conjugate used in the method of the invention may comprise 1 to 100, preferable 1 to 20 antigen recognizing moieties A, even more preferably 1 to 5 antigen recognizing moieties A (i.e. n = 1 to 100, preferable n = 1 - 20). The interaction of the antigen recognizing moiety with the target moiety can be of high or low affinity. Binding interactions of a single low-affinity antigen recognizing moiety may be too low to provide a stable bond with the antigen. Low-affinity antigen recognizing moieties can therefore be multimerized to furnish high avidity.

Preferable, the term “antigen recognizing moiety A” refers to an antibody directed against antigen expressed by the biological specimens (target cells) intracellular, like IL2, FoxP3, CD154, or extracellular, like CD3, CD14, CD4, CD8, CD19, CD25, CD34, CD45, CD56, and CD133, CD8alpha, CD90.2, CD90.1 for either human or mouse specimens.

The antigen recognizing moieties A, especially antibodies, can be coupled to the spacer P through side chain amino or sulfhydryl groups. In some cases, the glyosidic side chain of the antibody can be oxidized by periodate resulting in aldehyde functional groups.

The antigen recognizing moiety A can be covalently or non-covalently coupled to B. Methods for covalent or non-covalent conjugation are known by persons skilled in the art and the same as mentioned for conjugation of the detection moiety X.

The method of the invention is especially useful for detection and/or isolation of specific cell types from complex mixtures and may comprise more than one sequential or parallel sequences of the steps a) - d). The method may use a variety of combinations of conjugates. For example, a conjugate may comprise antibodies specific for two different epitopes, like two different anti-CD34 antibodies. Different antigens may be addressed with different conjugates comprising different antibodies, for example, anti-CD4 and anti-CD8 for differentiation between two distinct T-cell-populations or anti-CD4 and anti-CD25 for determination of different cell subpopulations like regulatory T-cells.

### Variants of the Method

The method of the invention provides a high flexibility for the specific labeling with the conjugate and release of the conjugate providing a plurality of different detection strategies.

Any step can be monitored qualitatively or quantitatively according to the detection moieties Xₒ used or by other applicable quantitative or qualitative methods known by persons skilled in the art, e.g., by visual counting. This can be useful to determine the efficiency of the individual steps provided by the method of the invention. Furthermore, it is possible to label the sample of biological specimen in or after any of the steps a) - d) for qualitatively or quantitatively monitoring. Such methods for labeling are known by persons skilled in the art.

### Step A)

In order to detect different target moieties or the same target moiety by different detection moieties, different conjugates having the general formula (I) Aₙ - Bₘ ⋯ C_{q}-Xₒ can be provided, wherein the conjugates and its components, A, B, C, X, n, m, q, o have the same meaning, but can be the same or different kind and/or amount of antigen recognizing moiety A and/or binding moiety B and/or binding moiety C and/or detection moiety X.

It was further found that tag/anti-tag systems comprising thiamine/anti-thiamine can be combined with the known tag/anti-tag systems comprising Biotin/anti-Biotin as the systems do not interfere with each other. This adds a further degree of flexibility and allows for further labelling and detection strategies.

In another embodiment of the invention, at least two conjugates are provided, which share the general characteristics of general formula (I), but wherein the respective binding pairs of first and second binding moiety are only specific to each other and do not cross-react or recognize the respective other binding partner.

More specific, in an embodiment of the method of the invention, a second conjugate complex according to general formula (III)

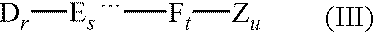

with D: antigen recognizing moiety;
E: first binding moiety
F: second binding moiety
Z: detection moiety;
r, s, t, u: integers between 1 and 100,
wherein E and F are non-covalently bound to each other and wherin E comprises a unit selected from the group consisting of Riboflavin (vitamin B2), Nicotinic acid (vitamin B3), Pantothenic acid (vitamin B5), Pyritinol, Biotin (vitamin B7), Folic acid (vitamin B9), Cyanocobalamin (vitamin B12), Pyridoxin (vitamin B6) and Ascorbic acid (vitamin C) and F is a moiety specifically recognizing E and antigen recognizing moiety D recognizes a different antigen as antigen recognizing moiety A is further provided in step a) and wherein a target moiety recognized by the antigen recognizing moiety D is labelled with at least one second conjugate in step b) and the thus labelled target moiety is detected via detecting moiety Y in step c) and the non-covalent bond between E and F is disrupeted by adding a release agent that binds to the first binding moiety E, thereby displacing the second binding moiety F and/or by adding a release agent that binds to the second binding moiety F, thereby displacing the first binding moiety E in step d).

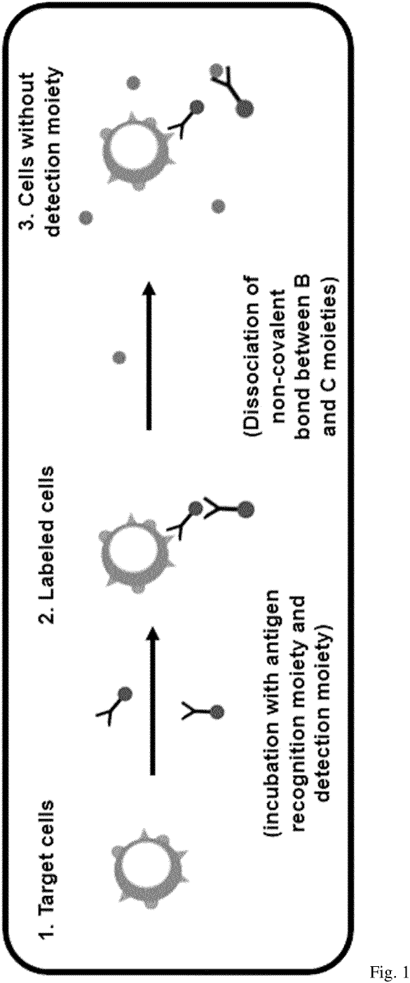

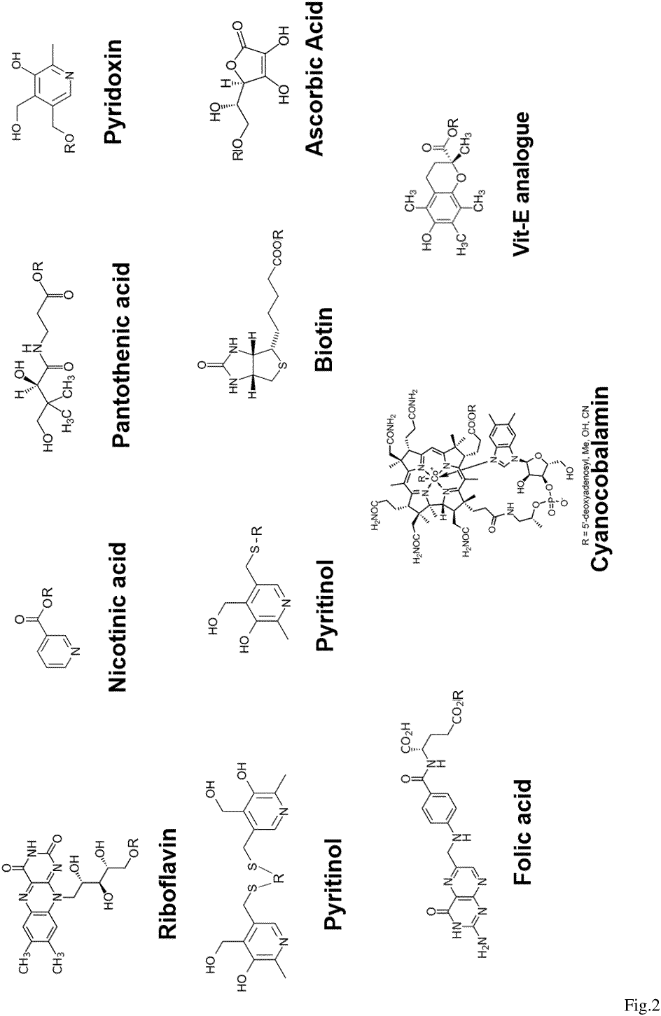

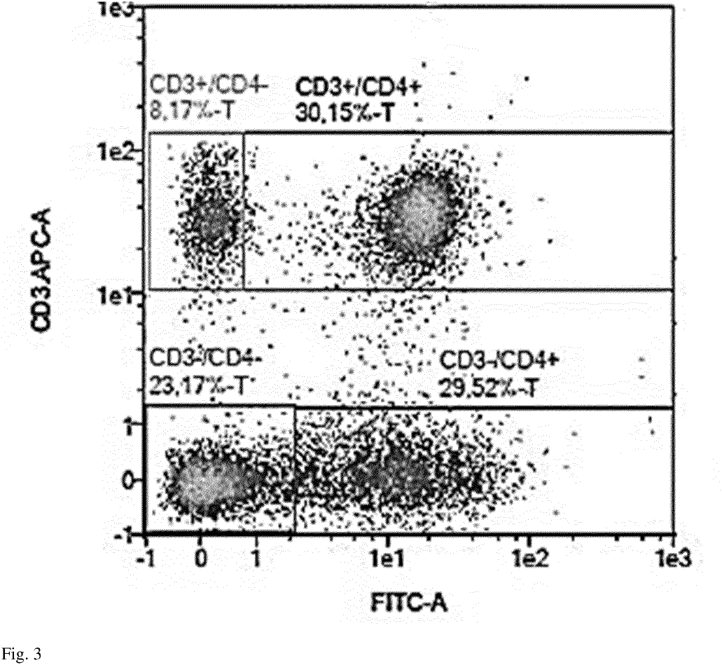

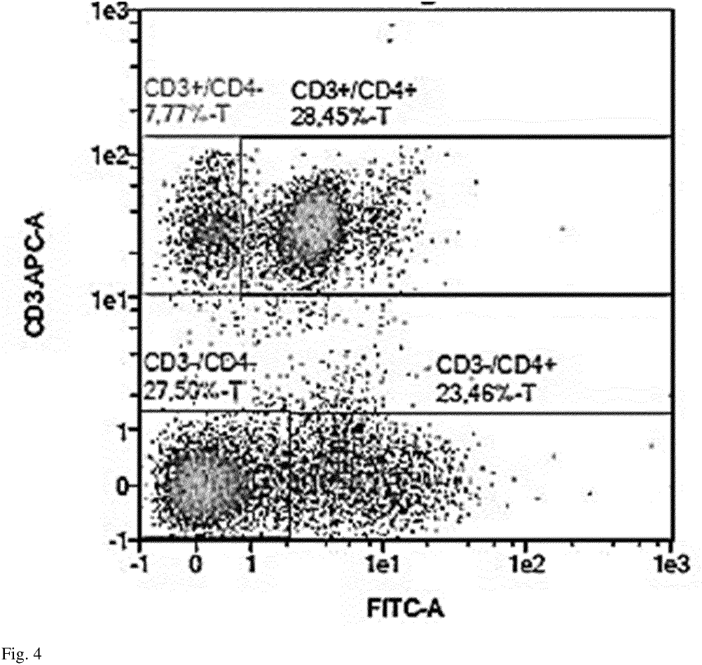

The formulas of first binding moieties B are shown in FIG. 1, wherin R stands for the covalent bond with antigen recognizing moiety D.

In this embodiment, conjugates according to general formula (I) and general formula (III) have to be provided with different release agents and/or steps d). It is possible to add the different release agents simultaneously or subsequently i.e. it is possible that one of the conjugates survives at least one of the cleaving steps d) and can be used for further detection or calibration purposes.

In a preferred embodiment, E and F stand for biotin or a biotin derivative and anti-biotin. Such conjugates and their release mechanism are known to the person skilled in the art.

### Step B)

In step b), the target moieties of the sample of biological specimens are labelled with the conjugates of the invention.

In a first embodiment of step b), labelling the target moiety with the conjugate is performed by first labelling the target moiety with a first conjugate An - Bm and second labelling the first labelled target moiety with a second conjugate Cq-Xo. In a variant of this embodiment, between the first and second labelling, a washing step is performed in order to reduce the amount of unbound An - Bm before the second incubation. Furthermore, after cleaving Cq-Xo from the labelled target moiety in step d) the target moiety labelled with An -Bm is labelled with a second conjugate Cq-Xo.

In a second embodiment of step b), the target moiety is labelled with the conjugate Aₙ - Bₘ ⋯ C_{q}-Xₒ directly, i.e. the assembly of the conjugate is performed before contacting with the target moiety recognized by the antigen recognizing moiety A.

Conditions during incubation are known by persons skilled in the art and may be empirically optimized in terms of time, temperature, pH, etc. Usually incubation time is up to 1h, more usually up to 30 min and preferred up to 15 min. Temperature is usually 4-37° C., more usually less than 37° C. and close to room temperature.

### Step C)

The method and equipment to detect the target moiety labeled with the conjugate Aₙ - Bₘ ⋯ C_{q}-Xₒ in c) is determined by the detection moiety X.

The method of the invention may be utilized not only for detecting target moieties i.e. target cells expressing such target moieties, but also for isolating the target cells from a sample of biological specimens according to the detection moiety X. In the method of the invention the term “detection” encompasses “isolation”.

For example, the detection of a target moiety by fluorescence may be used to trigger an appropriate separation process as performed on FACS or TYTO separation systems. In the method of the invention, the well-known magnetic cell separation process can also be used as detection and isolation process, wherein the magnetic particles are detected by the magnetic field.

In one variant of the invention, the detection moiety X is a fluorescent moiety. Targets labeled with fluorochrome-conjugate are detected by exciting the fluorescent moiety X and analyzing the resulting fluorescence signal. The wavelength of the excitation is usually selected according to the absorption maximum of the fluorescent moiety X and provided by LASER or LED sources as known in the art. If several different detection moieties X are used for multiple color/parameter detection, care should be taken to select fluorescent moieties having not overlapping absorption spectra, at least not overlapping absorption maxima. In case of a fluorescent moieties as detection moiety the targets may be detected, e.g., under a fluorescence microscope, in a flow cytometer, a spectrofluorometer, or a fluorescence scanner. Light emitted by chemoluminescence can be detected by similar instrumentation omitting the excitation.

In another variant of the invention the detection moiety is a light absorbing moiety, which is detected by the difference between the irradiation light intensity and the transmitted or reflected light intensity. Light absorbing moieties might also be detected by photoacoustic imaging, which uses the absorption of a pulsed laser beam to generate an acoustic like an ultrasonic signal.

Radioactive detection moieties are detected though the radiation emitted by the radioactive isotopes. Suitable instrumentation for detection of radioactive radiation include, for example, scintillation counters. In case of beta emission electron microscopy can also be used for detection.

Transition metal isotope mass tag moieties are detected by mass spectrometric methods such as ICP-MS, which is integrated in mass cytometry instrumentation.

In a further variant of the invention the detection moiety is a solid support. Depending on the size and density those might be detected by visual inspection or in a microscope.

Magnetic particles are detected magnetically, e.g., by magnetic relaxometry, magnetic resonance imaging (MRI), magnetic force microscopy (MFM), superconducting quantum interference devices (SQUIDs), magnetometer.

The target moiety can be isolated according to their detection signal by optical means, electrostatic forces, piezoelectric forces, mechanical separation, acoustic means or magnetic forces.

In one variant of the invention, suitable for such separations according to a fluorescence signal are especially flow sorters, e.g., FACS or MEMS-based cell sorter systems, for example as disclosed in EP14187215.0 or EP14187214.3.

In another variant, wherein the detection moiety is a solid support the isolation may be performed by mechanical trapping of the solid support, e.g., in a column or a sieve, or according to their density, e.g. by sedimentation or centrifugation.

Furthermore, target moieties labelled with a magnetic particle may be isolated by applying a magnetic field. Magnetic cell sorting is known to the person skilled in the art and can be conducted in a permanent or an electromagnetic field with or without the use of a ferromagnetic column containing ferromagnetic material. Columns containing ferromagnetic material enhance the gradient of the magnetic field and are available from Miltenyi Biotec B.V. & Co. KG, Germany.

Furthermore, during or after isolation of the target moieties contaminating non-labelled moieties of the sample of biological specimen can be removed by washing for example with buffer.

### Step D)

After detection step c), the non-covalent bond between Bₘ and C_{q} is disrupted in step d), thereby cleaving the binding moiety C_{q} and detection moiety Xₒ from the conjugate (I).

Disrupting the non-covalent bond between Bm and Cq in step d) may be performed by adding a release agent that binds to the first binding moiety B, thereby displacing the second binding moiety C and/or by adding a release agent that binds to the second binding moiety C, thereby displacing the first binding moiety B.

In a variant of the invention, this disruption step can be performed outside the detection system, e.g., in a solution of the target moiety. For example, target moieties labelled with a fluorescent or light absorbing moiety are incubated for disruption in a tube, or target moieties labelled with magnetic particles may be washed from the separation column and get the magnetic label removed outside of the magnetic field.

In another variant, the disruption step can implemented in the detection setup. For example, the disruption may take place during the detection of the signal, e.g., during fluorescence microscopy, cytometry, photometry or MRI. The reduction of the detection signal might therefore be monitored in real time. In another example the disruption may also take place within the magnetic field. The magnetically labeled target moiety can be unlabeled by adding, e.g. the competing molecule, to the column located in the magnetic field. In this variant, the target moieties are eluated from the column/the magnetic fields whereas the magnetic label remains on the column and in the magnetic field.

Optionally after disruption in d) there can be another step c) with detecting or isolating the target moiety.

The binding moiety C and detection moiety X and/or residual target moieties still labelled with the conjugate (I) or non-cleaved parts of conjugate (I) and/or the reagent used for disruption in d) can be separated from the sample. This kind of isolation step can be performed by a washing step or by utilizing the methods described in step c) detection and/or isolation. The separation can be achieved by mechanical trapping of the solid support, e.g., in a column or a sieve. Furthermore, magnetic particles as solid support can be removed by applying a magnetic field as already described for isolation of target moieties. Using ferromagnetic columns, this is preferable conducted in at least one (the same) or especially in two different columns containing ferromagnetic material.

The one or more optionally detection and/or isolation steps provide a possibility to separate the released target moiety or determine the efficiency of the disruption step d).

### Sequences of Steps A) to D)

The method of the invention may be performed in one or more sequences of the steps a) to d). After each sequence, the detection moiety and optionally the antigen recognizing moiety is released (removed) from the target moiety. Furthermore, sequences with combinations of any of the steps a) to d) are possible.

The method of the invention can be performed in the following embodiments:

Embodiment A of the invention is characterized in that steps a) to d) are performed in at least two subsequent sequences, wherein in each sequence conjugates Aₙ - Bₘ ⋯ C_{q}-X1ₒ (I) are used having different detection moieties X.

In this embodiment, the sample of biological specimens is contacted in a first step b) with a first conjugate Aₙ - Bₘ ⋯ C_{q}-X1ₒ, performing the detection in step c), cleaving the conjugate in step d). The sample of biological specimens (still labelled with A) is contacted in a next sequence with a second conjugate Aₙ - Bₘ ⋯ C_{q}-X2ₒ, the detection is performed, and the conjugate cleaved in step d). A, B, C are the same kind; n, m, q, o can be the same or different. This variant can be extended with further conjugates providing different X. An example for this variant is the magnetic labelling and isolation of a target cell population out of a sample of biological specimen followed by fluorescent labelling enabling flow cytometry or microscopy analysis or a fluorescent based flow sorting for further purification. In this variant the labelling efficiency in the second step can be reduced due to residual labelling with A after the first sequence.

Embodiment B of the invention is characterized in that step a) at least two conjugates Aₙ - Bₘ ⋯ C_{q}-Xₒ having different detection moieties X are provided and in at least two steps c) the labelled target moieties are detected via the different detection moieties X.

In this embodiment, the sample of biological specimens is contacted in step b) with a first conjugate Aₙ - Bₘ ⋯ C_{q}-X1ₒ and a second conjugate Aₙ - Bₘ ⋯ C_{q}-X2ₒ, performing the detection in two subsequent step c), cleaving both conjugates simultaneously in step d). A, B, C are the same kind; n, m, q, o can be the same or different. This variant can be extended with further conjugates providing different X. An example for this variant is the magnetic and fluorescent labelling and isolation of a target cell population out of a sample of biological specimen by magnetic cell separation followed by flow cytometry or microscopy analysis or a fluorescent based flow sorting for further purification. In this variant the labelling efficiency with X1 and X2 is reduced due to double labelling with the same antigen recognizing moiety.

Embodiment C of the invention is characterized in that in a first step a) at least one first conjugate Aₙ - Bₘ ⋯ C_{q}-X1ₒ is provided and after cleaving Cq-X1 from the labelled target moiety in a first step d), the target moiety still labelled with An - Bm is labelled with at least one second conjugate Cq-X2 in at least one second step a), wherein the first and second conjugates are provided with different detection moieties X.

In this embodiment, the sample of biological specimens is contacted in step b) with a first conjugate Aₙ - Bₘ ⋯ C_{q}-X1ₒ, performing the detection in step c), cleaving the conjugate in step d) therefore releasing C_{q}-X1ₒ. The sample of biological specimens still labelled with Aₙ - Bₘ is contacted in a next sequence with C_{q}-X2ₒ, the detection is performed, and the conjugate cleaved in step d) enabling a further sequence or cleaved in subsequent or simultaneous steps d). A, B, C are the same kind; n, m, q, o can be the same or different. An example for this variant is the magnetic labelling and isolation of a target cell population out of a sample of biological specimen followed by fluorescent labelling enabling flow cytometry or microscopy analysis or a fluorescent based flow sorting for further purification. Compared to embodiment A and B in this variant the labelling efficiency in the second step is not reduced due to readdressing of the same Aₙ - Bₘ of the first sequence.

Embodiment D of the invention is characterized in that steps a) to d) are performed in at least two subsequent sequences, wherein in each sequence conjugates Aₙ - Bₘ ⋯ C_{q}-X1ₒ (I) are used having different antigen recognizing moieties A and the same or different detection moieties X.

In this embodiment, the sample of biological specimens is contacted in step b) with a first conjugate Aₙ - Bₘ ⋯ C_{q}-X1ₒ, performing the detection in step c), cleaving the conjugate in step d). The sample of biological specimens or the isolated fraction of the first sequence is contacted in a next sequence with a second conjugate Aₙ - Bₘ ⋯ C_{q}-X2ₒ, the detection is performed, and the conjugate cleaved in step d). B, C are the same kind; n, m, q, o can be the same or different. X1 and X2 is the same or different. A first example for this variant is the sequential magnetic labelling and isolation of two target cell population out of a sample of biological specimen with cell populations recognized by only A1 and only A2. A second example is the magnetic labelling and isolation of a first target cell population and the fluorescent labelling and detection of a second target cell population out of a sample of biological specimen with cell populations recognized by only A1, only A2. A third example for this variant is the magnetic labelling and isolation of a target cell subpopulation recognized by A1+A2 out of a sample of biological specimen with cell populations recognized by only A1, only A2 and A1+A2.

After the first sequence with such systems, the sample of the biological specimen is still labelled with A1ₙ - Bₘ leading in the second sequence to a double labelling with A1ₙ - Bₘ ⋯ C_{q}-Xₒ and A2ₙ - Bₘ ⋯ C_{q}-Xₒ and to an isolation of all cell populations recognized by only A1, only A2 and A1/A2.

Embodiment E of the invention is characterized in that in step a) at least two conjugates Aₙ - Bₘ ⋯ C_{q}-Xₒ (I) having different antigen recognizing moieties A are provided and in step c) the labelled target moieties are detected via the same detection moiety X.

In this embodiment, the sample of biological specimens is contacted in step b) with a first conjugate A1ₙ - Bₘ ⋯ C_{q}-X1ₒ and a second conjugate A2ₙ - Bₘ ⋯ C_{q}-X1ₒ, performing the detection in step c), cleaving both conjugates in step d). B, C, X1 are the same kind; n, m, q, o can be the same or different. A first example for this variant is the simultaneous magnetic labelling and isolation of two target cell population out of a sample of biological specimen with cell populations recognized by only A1, only A2 or A1+A2. This variant can be extended with further conjugates providing different A. This variant can also be combined with variant C by cleaving the conjugates in step d) therefore releasing C_{q}-X1ₒ. The sample of biological specimens still labelled with A1ₙ-Bₘ respectively A2ₙ-Bₘ is contacted in a next sequence with Cq-X2o, etc. B, C are the same kind; n, m, q, o can be the same or different.

Embodiment F of the invention is characterized in that in step a) at least two conjugates Aₙ - Bₘ ⋯ C_{q}-Xₒ (I) having different antigen recognizing moieties A and different first binding moieties B or second binding moieties C are provided, and wherein the fragments Cq-Xo are cleaved from the target moieties labelled by the different conjugates by disrupting the non-covalent bond between Bm and Cq in separate steps d).

In this embodiment, the sample of biological specimens is contacted in step b) with a first conjugate A1ₙ - B1ₘ ⋯ C1_{q}-X1ₒ and a second conjugate A2ₙ - B2ₘ ⋯ C2_{q}-X1ₒ, performing the detection in step c), cleaving the conjugates in step d) wherein step d) is different for B1-C1 and B2-C2. P, X are the same kind; n, m, q, o can be the same or different. This variant can be extended with further conjugates of general formula ANn-P-BNm-CNq-Xo with N between 1 and 10. An example for this variant is the simultaneous magnetic labelling and isolation of two target cell population out of a sample of biological specimen with at least two different target cell populations recognized by only A1 and only A2 respectively.

Embodiment G of the invention is characterized in that in step a) at least two conjugates Aₙ - Bₘ ⋯ C_{q}-Xₒ (I) having different antigen recognizing moieties A, different detection moieties X and different first binding moieties B or second binding moieties C are provided, and wherein the fragments Cq-Xo are cleaved from the target moieties labelled by the different conjugates by disrupting the non-covalent bond between Bm and Cq in separate steps d).

In this embodiment, the sample of biological specimens is contacted in step b) with a first conjugate A1ₙ - B1ₘ ⋯ C1_{q}-X1ₒ and a second conjugate A2ₙ - B2ₘ ⋯ C2_{q}-X2ₒ, performing the detection simultaneously or in two subsequent steps c), the conjugates in subsequent or simultaneous steps d) and e) wherein step d) is different for B1-C1 and B2-C2. n, m, q, o can be the same or different. An example for this variant is the fluorescent labelling with two parameters and two fluorescent dye and isolation of a target cell subpopulation out of a sample of biological specimen with cell populations recognized by only A1, only A2 and A1+A2. The different C1_{q}-X1ₒ and C2_{q}-X2ₒ enable a specific labelling and the release can be performed simultaneously or sequentially in different steps d). This variant can be extended with further conjugates providing different A and X, e.g., for multiple parameter fluorescent labelling, detection, isolation and release of cell subpopulations. This variant can also be combined with variant C.

### Use of the Method

The method of the invention can be used for various applications in research, diagnostics and cell therapy.

In a first use of the invention, biological specimens like cells are detected or isolated for counting purposes i.e. to establish the amount of cells from a sample having a certain set of antigens recognized by the antigen recognizing moieties of the conjugate.

In a second use, one or more populations of biological specimens are separated for purification of target cells. Those isolated purified cells can be used in a plurality of downstream applications like molecular diagnostics, cell cultivation, or immunotherapy.

In other uses of the invention, the location of the target moieties like antigens on the biological specimens recognized by the antigen recognizing moieties of the conjugate is determined. Advanced imaging methods are known as “Multi Epitope Ligand Cartography”, “Chip-based Cytometry” or “Multioymx” and are described, for example, in EP 0810428, EP1181525, EP 1136822 or EP1224472. In this technology, samples of biological specimen are contacted in sequential cycles with antigen recognizing moieties coupled to a detection moiety, the location of the antigen is detected by the detection moiety and the detection moiety is afterwards eliminated. Therefore, subsequent cycle of labelling-detection-elimination provide the possibility to map protein networks, localize different cell types or the analysis of disease-related changes in the proteome.

### EXAMPLES

### Example 1

Human peripheral blood mononuclear cells (PBMC) cells (ca. 2E6 cells, 100 µL) were incubated (10 min at 4° C.) with 1 µg/mL of anti-CD4 recombinant Antibody labeled with Thiamine. The sample was then washed twice with 1 mL of PEB buffer and cells centrifuged at 2000 rpm fro 5 min. After washing, the sample was resuspended in 100 µL of PEB buffer. The CD4-Thiamine labeled cells were then incubated with anti-Thiamine antibody (Polyclonal Rat antibody obtained from Abcam plc, product ID: ab37125) to a final concentration of 10 µg/mL and incubated for 10 minutes at 4° C. As before, the sample was washed twice by centrifugation with PEB buffer and then resuspended to a final volume of 100 µL. The sample was then split into 25 µL aliquots were one was used for direct staining and a second one for Thiamin release. The direct staining aliquot was processed as follows: cells were labeled with Goat-anti-Rat IgG-FITC antibody conjugate (10 min 4° C.) which should bind the Fc region of the anti-Thiamine antibody, as well as with CD3-APC for counterstain of T cells. The sample was washed by centrifugation, supernatant decanted and resuspended in 300 µL of PEB buffer. The thiamine release aliquot was processed as follows: cells were incubated with 2 mM of free Thiamine solution in PEB buffer for 10 min at RT. The sample was then washed by centrifugation and cell resuspended in 50 µL of PEB buffer. Cells were then stained Rat-anti-Mouse IgG1-FITC and CD3-APC, incubated 10 min at 4° C., washed by centrifugation, supernatant discarded and cells resuspended in 300 µL of PEB buffer. All samples were then measured directly using a MACSquant analyzer, and gated on Lymphocytes and then living cells, to obtain IgG-FITC vs CD3-APC cells to define the MFI for the target populations. The direct stain aliquot shows an average MFI value of 17.82 with a SD of 5.58. The thiamine release aliquot shows an average MFI value of 3.28 with a SD of 1.19. This indicates that upon incubation with free Thiamine the binding between CD4-Thiamine and anti-Thiamine antibody is partially loss, as indicated by a significant loss of the average MFI, which represents ca. 81.6% of release.

1. Conjugate complex for detecting a target moiety in a sample of biological specimens having the general formula (I) 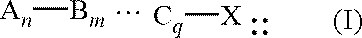
with A: antigen recognizing moiety;
B: first binding moiety
C second binding moiety
X: detection moiety;
n, m, q, o integers between 1 and 100,
wherein B and C are non-covalently bound to each other characterised in that B comprises a thiamine unit and C is a moiety recognizing thiamine.

2. Conjugate according to claim 1 characterised in that first binding moiety B comprises a thiamine unit according to formula IIa 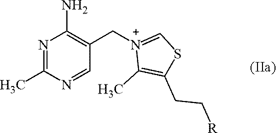
With R representing a spacer group selected from the group consisting of LC, LCLC, PEGn, peptide, amino acid, protein, antibody, antibody fragment, fluorescent protein, nanobody and interleukine.

3. Conjugate according to claim 1 or 2 characterised in that moiety C recognizing thiamine is an antibody, a fragmented antibody or fragmented antibody derivative.

4. Method for detecting a target moiety in a sample of biological specimens by:
a) providing at least one conjugate according to any of the claims 1 to 3 to the sample
b) labelling the target moiety recognized by the antigen recognizing moiety A with at least one conjugate
c) detecting the labelled target moiety via detecting moiety X
d) disrupting the non-covalent bond between Bₘ and C_{q} by adding a release agent that binds to the first binding moiety B, thereby displacing the second binding moiety C and/or by adding a release agent that binds to the second binding moiety C, thereby displacing the first binding moiety B.

5. Method according claim 4, characterized in that labelling the target moiety with the conjugate is performed in step b) by labelling the target moiety with a conjugate Aₙ - Bₘ ... C_{q}-X (I).

6. Method according to claim 4, characterized that labelling the target moiety in step b) with the conjugate is performed by first labelling the target moiety with a first conjugate Aₙ - Bₘ and second labelling the first labelled target moiety with a second conjugate C_{q-}X.

7. Method according to claim 6, characterized that between the first and second labelling, a washing step is performed.

8. Method according to claim 4, characterized in that steps a) to e) are performed in at least two subsequent sequences, wherein in each sequence conjugates Aₙ - Bₘ ... Cq-X (I) are used having different detection moieties X.

9. Method according to claim 4, characterized in that step a) at least two conjugates Aₙ - Bₘ ... C_{q}-X having different detection moieties X are provided and in at least two steps c) the labelled target moieties are detected via the different detection moieties X.

10. Method according to claim 4, characterised in that a second conjugate complex according to general formula (III) 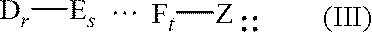
with D: antigen recognizing moiety;
E: first binding moiety
F: second binding moiety
Z: detection moiety;
r, s, t, u: integers between 1 and 100,
wherein E and F are non-covalently bound to each other
and wherin E comprises a unit selected from the group consisting of Riboflavin (vitamin B2), Nicotinic acid (vitamin B3), Pantothenic acid (vitamin B5), Pyritinol, Biotin (vitamin B7), Folic acid (vitamin B9), Cyanocobalamin (vitamin B12), Pyridoxin (vitamin B6) and Ascorbic acid (vitamin C) and F is a moiety specifically recognizing E and antigen recognizing moiety D recognizes a different antigen as antigen recognizing moiety A is further provided in step a) and wherein a target moiety recognized by the antigen recognizing moiety D is labelled with at least one second conjugate in step b) and the thus labelled target moiety is detected via detecting moiety Y in step c) and the non-covalent bond between E and F is disrupted by adding a release agent that binds to the first binding moiety E, thereby displacing the second binding moiety F and/or by adding a release agent that binds to the second binding moiety F, thereby displacing the first binding moiety E in step d).

11. Method according to claim 10, characterized in that E comprises biotin and F is a biotin recognizing moiety.
